(19) United States
(12) Patent Application Publication
(10) Pub. No.: US 2025/0152229 A1
(43) Pub. Date: May 15, 2025

(54) Interface Joint

(71) Applicant: CREO MEDICAL LTD, Chepstow South Wales (GB)

(72) Inventors: George Ullrich, Gethesda Gwynedd (GB); David Webb, Gethesda Gwynedd (GB)

(73) Assignee: CREO MEDICAL LTD, Chepstow South Wales (GB)

(21) Appl. No.: 18/876,628

(22) PCT Filed: Jun. 16, 2023
(86) PCT No.: PCT/EP2023/066261

§ 371 Date: Dec. 18, 2024

(30) Foreign Application Priority Data
Jun. 17, 2022 (EP) 2208971.8

(51) Int. Cl. A61B 18/12; A61B 18/00

(52) U.S. Cl. CPC A61B 18/1206; A61B 2018/00178; A61B 2218/002

(57) ABSTRACT

Various embodiments provide an interface joint for interconnecting an electrosurgical generator, a fluid supply, and an electrosurgical instrument. The interface joint comprises a housing having: an electrical inlet for receiving radiofrequency (RF) electromagnetic (EM) energy and/or microwave frequency EM energy from the electrosurgical generator; a fluid inlet for receiving fluid from the fluid supply; and, an outlet. The interface joint also comprises a single cable assembly for connecting the outlet to an instrument tip of the electrosurgical instrument. The single cable assembly comprises: a flexible shaft having: a coaxial cable that is connected to the electrical inlet; and a fluid channel that is in fluid communication with the fluid inlet. The housing includes a first portion and a second portion, the second portion being attached to the single cable assembly such that rotation of the second portion effects rotation of the single cable assembly; and the first portion being rotatable relative to the second portion. The first portion includes a first inlet, and the second portion includes a second inlet, wherein the first inlet is one of the fluid inlet or electrical inlet, and the second inlet is the other of the fluid inlet or electrical inlet.

### FIELD OF THE INVENTION

The present invention relates to an interface joint for interconnecting an electrosurgical instrument to a fluid supply and an electrosurgical generator. The electrosurgical instrument may be for delivering radiofrequency and/or microwave frequency energy into biological tissue.

### BACKGROUND

Surgical resection is a means of removing sections of organs from within the human or animal body. Such organs may be highly vascular. When tissue is cut (divided or transected) small blood vessels called arterioles are damaged or ruptured. Initial bleeding is followed by a coagulation cascade where the blood is turned into a clot in an attempt to plug the bleeding point. During an operation, it is desirable for a patient to lose as little blood as possible, so various devices have been developed in an attempt to provide blood free cutting. For endoscopic procedures, bleeds are also undesirable, and need to be dealt with in an expedient manner, since the blood flow may obscure the operator's vision, which may prolong surgery and potentially lead to the procedure needing to be terminated and another method used instead, e.g. open surgery.

Electrosurgical generators are prevalent in hospital operating theatres, often for use in open and laparoscopic procedures, and increasingly for use with surgical scoping devices, e.g. an endoscope or the like. In endoscopic procedures the electrosurgical accessory is typically inserted through a lumen inside an endoscope. Considered against the equivalent access channel for laparoscopic surgery, such a lumen is comparatively narrow in bore and greater in length.

Instead of a sharp blade, it is known to use radiofrequency (RF) energy to cut biological tissue. The method of cutting using RF energy operates using the principle that as an electric current passes through a tissue matrix (aided by the ionic contents of the cells and the intercellular electrolytes), the impedance to the flow of electrons across the tissue generates heat. In practice, an instrument is arranged to apply an RF voltage across the tissue matrix that is sufficient to generate heat within the cells to vaporise the water content of the tissue. However, as a result of this increasing desiccation, particularly adjacent to the RF emitting region of the instrument (which has the highest current density of the current path through tissue), direct physical contact between the tissue and instrument can be lost. The applied voltage then manifests itself as a voltage drop across this small void, which causes ionisation in the void that leads to a plasma. Plasma has a very high volume resistivity compared with tissue. The energy supplied to the instrument maintains the plasma, i.e. completes the electrical circuit between the instrument and the tissue. Volatile material entering the plasma can be vaporised and the perception is therefore of a tissue dissecting plasma.

GB 2 523 246 describes an interface joint for integrating into a single cable assembly all of (i) a fluid feed, (ii) a needle movement mechanism, and (iii) an energy feed (e.g. a cable supplying RF and/or microwave energy). The cable assembly may be sized to fit through the instrument channel of a conventional endoscope. Also described is a torque transfer unit for permitting controlled rotation of the cable assembly within the instrument channel of the endoscope. The interface joint and torque transfer unit may be integrated as a single component. The interface joint may rotate with the torque transfer unit under the control of a user.

The present invention has been devised in light of the above considerations.

### SUMMARY OF THE INVENTION

The present invention provides a development to the concept discussed in GB 2 523 246.

At its most general, the inventors have developed a modified interface joint which may integrate into a single cable assembly a fluid feed and an energy feed (e.g. a cable supplying RF and/or microwave energy) for delivery to an electrosurgical instrument. The single cable assembly may be sized to fit through the instrument channel of a conventional endoscope.

According to a first aspect of the invention, there is provided an interface joint for interconnecting an electrosurgical generator, a fluid supply, and an electrosurgical instrument, the interface joint comprising: a housing having: an electrical inlet for receiving radiofrequency (RF) electromagnetic (EM) energy and/or microwave frequency EM energy from the electrosurgical generator; a fluid inlet for receiving fluid from the fluid supply; and an outlet; and a single cable assembly for connecting the outlet to an instrument tip of the electrosurgical instrument, the single cable assembly comprising a flexible shaft having: a coaxial cable that is connected to the electrical inlet; and a fluid channel that is in fluid communication with the fluid inlet; wherein the housing includes a first portion and a second portion, the second portion being attached to the single cable assembly such that rotation of the second portion effects rotation of the single cable assembly; and the first portion being rotatable relative to the second portion; wherein the first portion includes a first inlet and the second portion includes a second inlet, wherein the first inlet is one of the fluid inlet or electrical inlet, and the second inlet is the other of the fluid inlet or electrical inlet.

This arrangement provides several advantages which may improve the manoeuvrability and rotational control of the electrosurgical instrument. Since the housing includes a first and second portion which are rotatable relative to each other, the second portion may be rotated to effect rotation of instrument tip, without causing the first portion to rotate as well. Therefore, the first portion may remain relatively static during rotation of the instrument tip, and may be held (e.g. by the surgeon's spare hand or by an assistant) to help stabilise and control the interface joint without in turn inhibiting rotation of the second portion and thus the instrument tip. Additionally, by providing the fluid inlet and electrical inlet at independently rotatable (first and second) portions of the housing, the interface joint can mitigate any risk of feed cables tangling around each other or around the interface joint.

Since the first portion may be held without inhibiting rotation of the second portion, the first portion may also be referred to as a ‘handle portion’. The second portion may be referred to herein as comprising a ‘torquer portion’, ‘torquer’, or ‘integrated torque transfer unit’. As will be discussed further herein, the second portion may also be referred to herein as having a branched portion within which the fluid and EM energy are integrated into the single cable assembly (or into a tubular member for conveying to the single cable assembly). In some embodiments, the branched portion may be integral with (and may therefore rotate with) the torquer portion, which may rotate together relative to the first (handle) portion. In other embodiments, the branched portion may be rotatable relative to the torquer portion as well as relative to the first (handle) portion. These arrangements will be discussed further herein.

In general, since the second portion may effect rotation of the single cable assembly while the first portion may be held, the second portion may be described herein as being ‘rotatable’ while the first portion may be described as being ‘static’. However, these terms are used purely for convenience, using a frame of reference in which the first portion is static. It will be appreciated that in reality, each portion may rotate, albeit independently of one another.

The first and second portions may be coupled to each other by a free-rotating connector, e.g. by a rotatable snap-fit connection. Optionally, the interface joint may include a low-friction element between the first and second portions. For example, the low-friction element may comprise a polymer O-ring (e.g. PTFE O-ring) between the first and second portions. This may facilitate relative rotation of these portions, reducing the chance of any rotation (or lack thereof) at the first portion affecting rotation of the second portion (and thus the instrument tip).

Any portion of the interface joint (the first/handle portion, torquer portion, and/or branched portion) may be fully rotatable relative to another one of the portions, e.g. through 360 degrees. Alternatively, in some embodiments, the relative rotation between any of the portions may be restricted to a maximum threshold, e.g. up to 180 degrees, optionally 120 degrees, optionally 90 degrees. For example, optionally, the first portion and the second portion may only be rotatable through 180 degrees relative to each other. For example, the first portion may include a stopper element configured to restrict the relative rotation by abutting against a corresponding surface of the second portion to prevent relative rotation thereof. In an embodiment, the stopper element may be located on the second portion and the corresponding surface may be on the first portion. In an embodiment, the branched portion and the torquer portion may have a stopper element and a corresponding surface to restrict relative rotation.

Optionally, the interface joint may further comprise a fluid feed cable connected to the fluid inlet for conveying fluid from the fluid supply to the interface joint. Further, optionally, the interface joint may include an electrical feed cable connected to the electrical inlet for conveying the EM energy from the electrosurgical generator to the interface joint. Alternatively, the interface joint may be provided without the fluid feed cable and/or electrical feed cable, which may be supplied separately. As noted above, the independently rotatable first and second portions may help to reduce the risk of the fluid feed cable and/or electrical feed cable tangling around each other or around the interface joint.

Optionally, the fluid inlet may include a connector (e.g. a free-rotating fluid connector) for connecting to the fluid feed cable. Optionally, the electrical inlet may include a connector (e.g. a free-rotating electrical connector) for connecting to the electrical feed cable. Examples of suitable free-rotating connectors include QMA-type or MPX-type connectors. The use of a free-rotating connector may help allow the interface joint to be rotated relative to the cable(s), without causing excess tension or twisting of the cable(s).

The electrosurgical instrument may be any device which in use is arranged to use RF EM energy and/or microwave frequency EM energy for the treatment of biological tissue. The electrosurgical instrument may use the RF EM energy and/or microwave frequency EM energy for any or all of resection, coagulation and ablation. For example, the instrument may be a resection device as disclosed in GB 2 523 246 A or in GB application no 2119001.2, but alternatively may be any of a pair of microwave forceps, a snare that radiates microwave energy and/or couples RF energy, and an argon beam coagulator.

Herein, radiofrequency (RF) may mean a stable fixed frequency in the range 10 kHz to 300 MHz and microwave frequency may mean a stable fixed frequency in the range 300 MHz to 100 GHz. The RF energy should have a frequency high enough to prevent the energy from causing nerve stimulation and low enough to prevent the energy from causing tissue blanching or unnecessary thermal margin or damage to the tissue structure. Preferred spot frequencies for the RF energy include any one or more of: 100 KHz, 250 kHz, 400 KHz, 500 kHz, 1 MHZ, 5 MHz. Preferred spot frequencies for the microwave energy include 915 MHZ, 2.45 GHZ, 5.8 GHZ, 14.5 GHZ, 24 GHZ.

The electrosurgical generator may be any device capable of delivery RF EM energy or microwave frequency EM energy for treatment of biological tissue. For example, the generator described in WO 2012/076844 may be used.

The fluid supply may be a high-pressure fluid supply, for supplying high-pressure fluid to the instrument tip. Such high pressure arrangements will be discussed further herein.

The interface joint may be particularly suitable for gathering a plurality of inputs into a single cable assembly before it is inserted through the instrument channel of an endoscope. To achieve this, the cable assembly may have an outer diameter of 9 mm or less, e.g. 2.8 mm or less for a flexible video colonoscope. In some embodiments, the fluid channel of the single cable assembly may convey the coaxial cable therethrough. Alternatively, fluid may be conveyed through an insulated passage in the centre of the coaxial cable, e.g. as described in GB application no 2119001.2.

The first portion may be located at a proximal region of the interface joint, and the second portion may be located at a distal region of the interface joint. As used herein, the term “proximal” may refer to a region away from the instrument tip, whilst the term “distal” may refer to a region located closer to the instrument tip. The first portion and the second portion may each be sized to be held by a user's hand, e.g. to be gripped between a thumb and finger of a user. For example, the first portion and/or second portion may each have lengths of 3 cm or more, e.g. between 3-10 cm, e.g. between 5-8 cm. The housing may comprise (e.g. consist of) electrically insulating material. For example, the first and second portions may each comprise (e.g. consist of) electrically insulating material.

Optionally, the first portion and/or the second portion may include a gripping surface, for example, so that it/they can be more securely held by a user. The gripping surface may comprise one or more indentations, protrusions, and/or a concave profile sized to facilitate grip by the user. For example, the gripping surface may be sized to be gripped by the thumb and forefinger of the user. By providing one or more gripping surfaces in combination with independently rotatable first and second portions, the interface joint may further help to enable independent manoeuvrability of the first and second portions.

Optionally, the second portion may comprise a conduit defining a branched passageway. The branched passageway may have a first length in line with the first inlet and the outlet, and a second length having the second inlet. By having a branched passageway that provides an in-line connection between the first inlet and the outlet, the interface joint can reduce the risk of a feed cable connected to the first inlet wrapping around and restricting movement of the interface joint.

As used herein, the phrase “in-line” may refer to a connection in which the first inlet is oriented along (or parallel to) a longitudinal axis of the interface joint. For example, the first inlet may be located at a proximal end surface of the first portion, and the outlet may be located at a distal end surface of the second portion (opposite the proximal end surface).

The branched passageway may have any suitable configuration. For example, the first length may terminate at its proximal end with the first inlet (or with a port connected thereto), and the second length may terminate at its end with the second inlet. Optionally, the second length may extend at an angle to the first length. The second inlet may therefore be angled relative to (i.e. non-parallel to) the first inlet. This may help to further avoid tangling of any feed cables at the first and second inlets, since the cables will feed into the device from different directions. In variant embodiments, which are not illustrated, the first length and second length may run substantially parallel to each other (with the first inlet being parallel to the second inlet) before combining into a single passage extending toward the outlet, e.g. in a type of Y-shaped conduit.

Optionally, the second length may extend at a substantially perpendicular angle to the first length. A substantially perpendicular angle may help to reduce the risk of tangling between the fluid feed cable, EM feed cable, and single cable assembly, by keeping the cables angled as far apart as possible.

As used herein, “substantially” as used with reference to an angle may allow deviations of less than 15°, preferably less than 10°, more preferably less than 5°. Therefore, the second length may be angled between 75°-105° relative to the first length, preferably between 80°-100°, more preferably between 85°-95°. In perpendicular arrangements, the conduit may be referred to as a T-shaped conduit.

Optionally, the second length may extend at an acute angle to a proximal end of the first length. For example, the second length may extend at an angle 20°-60° relative to the first length, more preferably 30°-50°, more preferably 40°. In such arrangements, the conduit may be referred to as a type of ‘Y-shaped’ conduit. A terminal (proximal) end of the second length may therefore be closer to the proximal end of the first length than to the distal end of the first length. Consequently, a feed cable (e.g. fluid feed cable or EM feed cable) which is input into the second length, may be angled away from a distal end of the interface joint. This may help to mitigate any tangling or coiling at a distal end of the interface joint (which may comprise the torquer portion), thereby reducing the risk of obstructing rotation of the single cable assembly.

The housing may be an elongate capsule sized to fit in an operator's hand. The first length and second lengths may have different lengths or may have the same length. Optionally, the housing may provide a double isolation barrier for the operator, i.e. the housing may comprise an outer casing (first level of isolation) that encapsulates the conduit defining the branched passageway (second level of isolation) within which the various inputs are integrated into the single cable assembly. The branched passageway may provide a watertight volume (fluid-tight cavity) which defines a fluid flow path between the fluid inlet and the outlet, and which has an electrical port for admitting the coaxial cable through the watertight volume. Both the electrical port and the outlet may include one or more bungs (also referred to as “sealing bungs”) which define a watertight passage for the coaxial cable. The one or more sealing bungs may be formed from a resiliently deformable material, e.g. silicone rubber, whereby the coaxial cable is encapsulated in the material as it passes through it. Sealing the watertight volume in this way means that the only route for fluid out of the interface joint is through the outlet along the fluid channel in the single cable assembly.

Optionally, the first inlet is the electrical inlet, and the second inlet is the fluid inlet. This arrangement may provide several advantages, as discussed below.

Firstly, this may allow the electrical feed cable to remain relatively static during rotation of the single cable assembly, thereby mitigating any interference of the EM signal which could otherwise be caused by coiling-up or bending of the electrical feed cable around the interface joint (e.g. compared to an alternative arrangement in which the electrical inlet is comprised by and rotates with the second portion).

Secondly, in arrangements where the first inlet is in line with the outlet, this arrangement may provide a straight path for the coaxial cable through the housing, thereby further helping to avoid signal losses which could otherwise occur along a bent/coiled cable.

Thirdly, in arrangements where the branched conduit includes a second length extending at an angle to the first length, providing the fluid inlet along the second length may be particularly convenient since fluid can be freely injected into the branched passageway without requiring a fluid cable to carry the fluid and bend around the corner between the first and second lengths.

Optionally, the second portion includes a branched portion and a torquer portion, the branched portion being rotatable relative to the torquer portion, wherein the branched portion includes the second inlet and the torquer portion includes the outlet. In this manner, the fluid inlet, electrical inlet, and outlet may each be located in different portions of the housing which are independently rotatable relative to each other (i.e. the first portion, torquer portion, and branched portion). The fluid feed cable and coaxial feed cable can therefore each be independently positioned during rotation of the single cable assembly. Such an arrangement can further help to facilitate manoeuvrability and mitigate signal losses by mitigating any bending/coiling/tangling of feed cables during rotation of the instrument tip. For example, an independently rotatable branched portion may help ensure that the feed cable at the second inlet does not wrap around and restrict movement of the interface joint during rotation of the instrument tip.

The branched portion may include any of the conduits (e.g. Y-shaped or T-shaped conduits) discussed above for combining the fluid and EM energy to be conveyed to the single cable assembly.

The handle portion and torquer portion (which may be located at proximal and distal ends of the interface joint, respectively) may alternatively be referred to as a “proximal portion” and a “distal portion”, respectively. The rotatable branched portion, if present, may be located between the handle portion and the torquer portion, and may therefore be referred to as an “intermediate portion”.

Optionally, the branched portion is configured to combine the fluid and EM energy into a tubular member to be conveyed to the torquer portion, wherein the tubular member is rotatable relative to the branched portion. By providing a tubular member which is rotatable relative to the branched portion, the single cable assembly and/or tubular member may be rotated without rotating the branched portion. This may further help to improve manoeuvrability and EM energy/fluid transmission.

The tubular member may extend through and beyond the branched portion, to protrude from the distal end of the branched portion. In particular, the distal end of the tubular member may extend into the single cable assembly in the torquer portion. Rotation of the torquer portion may therefore effect rotation of the tubular member via the single cable assembly. A proximal end of the tubular member may connect to the electrical inlet, to receive the coaxial cable through the tubular member.

Optionally, the interface joint may further include one or more bungs (e.g. the sealing bungs discussed above) within the branched portion to form a fluid-tight seal around the tubular member. The one or more bungs may be provided at the distal and/or proximal ends of the branched portion. This may assist in sealing the watertight volume (fluid-tight cavity) between the fluid inlet and outlet and preventing leakage past the tubular member. The tubular member may be rotatable relative to the bung(s), whereas the bung(s) may be non-rotatable (fixed) relative to the branched portion. The tubular member and/or bung(s) may include a low-friction material to help facilitate relative rotation of these components. For example, the bung(s) and/or tubular member may be coated with an anti-friction coating or lubricant. Alternatively, the bung(s) may be formed of a low-friction material, e.g. silicone or polyisoprene rubber. Optionally, the bung(s) may be formed of a self-healing or resiliently deformable material. Advantageously, a self-healing or resiliently deformable material may help to form a tight seal around the tubular member, whilst also having relatively low friction.

Optionally, the tubular member is configured to convey the coaxial cable that is connected to the electrical inlet, and the tubular member includes one or more apertures for permitting fluid to flow into the tubular member. Accordingly, fluid may be conveyed into the tubular member (from the fluid inlet and through the fluid-tight cavity) and around the coaxial cable, thereby providing a simple mechanism for combining the fluid and EM energy into the single tubular member and subsequently into the single cable assembly.

The tubular member may comprise a hypotube. The hypo tube may be a metal (e.g. stainless steel) tube with a blunt end. The hypo tube may extend into the torquer portion and may therefore bridge a junction between the branched portion and the torquer portion. A metal hypotube may be relatively robust, and may be particularly useful for embodiments utilising high pressure fluid. In particular, in some embodiments, the electrosurgical instrument may omit a needle for puncturing tissue, and may instead utilise a high-pressure fluid which is pressurised for the purpose of puncturing or lifting tissue at a distal end of the instrument. Examples of suitable instruments which may operate in this manner are described in GB application no 2119001.2, which is incorporated herein by reference in its entirety. Therefore, optionally, the interface joint is not configured to integrate a needle movement mechanism into the single cable assembly. For example, optionally, the interface joint does not include a needle actuator (e.g. slidable trigger or pivoting trigger) on the housing, the needle actuator being attached to a push rod that extends out of the housing through the outlet for controlling a needle at the distal end of the instrument. By omitting a needle movement mechanism from the interface joint, the interface joint itself may be made more compact, and may be used in conjunction with instruments that do not utilise a needle and push rod (and can therefore also be more compact).

The electrosurgical instrument and interface joint may be used to convey fluid at a range of pressures, e.g. high pressures. For example, the instrument and interface joint may be used to convey fluid at pressures of at least 100 psi, optionally at least 150 psi, optionally at least 200 psi, optionally, at least 250 psi. The specific pressures used may be selected to take into account the tissue characteristics at a target region. For example, in order to pierce mucosa (e.g. for forming initial cuts around a lesion), higher pressures may be needed than to pierce submucosa (e.g. for topping-up the lesion with fluid once the mucosa has been pierced). For example, 100 psi may provide a useful pressure for piercing submucosal tissue. Additionally, tissues in some organs (e.g. the GI tract) may be easier to pierce than others (e.g. stomach) and may therefore require lower pressures. For example, the instrument may convey fluid in a range of 250-300 psi for piercing tissue in the lower GI tract, and fluid in the range of 400-500 psi for piercing tissue in the stomach. Further, the pressures may be controlled or reduced to avoid using pressures which are too high for a particular application or body region, e.g. in order to mitigate the risk of unintended perforation.

Optionally, two or more of the portions of the housing are releasably attachable to each other. Releasable attachment may be provided in any suitable manner, e.g. via rotatable snapfit connections. This may facilitate assembly and ease of use. For example, any two or more of the first portion, branched portion, and/or torquer portion may be releasably attachable to each other.

Optionally, the interface joint may further include a cable management structure for securing a section of a feed cable to the interface joint, the feed cable being either: a fluid feed cable for conveying fluid to the fluid inlet from the fluid supply; or an EM feed cable for conveying the EM energy from the electrosurgical generator to the electrical inlet. For example, the cable management structure may secure the feed cable to the housing of the interface joint, or to another feed cable of the interface joint.

The section of the feed cable may be any section of the feed cable which is spaced from an end of the feed cable that is attached to the interface joint. For example, this may be at a proximal section or mid-section of the feed cable. The cable management structure may comprise a clip or other attachment device for securing the section of the feed cable to the housing or to another feed cable. The cable management structure may help to ensure that any coiling of the feed cable occurs in a preferred direction relative to the interface joint. Further, the cable management structure may be used to secure the feed cable at a position which provides excess slack in a portion of the feed cable located between the cable management structure and the inlet. By providing this excess slack in the feed cable, manoeuvrability can be improved, since the feed cable may coil around the interface joint as the interface joint is rotated, without tightly winding against the interface joint and restricting it.

Optionally, the cable management structure may be attachable to the feed cable at a position which enables a predetermined number of rotations of the second portion relative to the first portion. For example, the cable management structure may be positioned to provide sufficient slack in the feed cable so as to permit two full rotations of the second portion relative to the first portion. This may help to improve the manoeuvrability of the instrument whilst also balancing this with the amount of excess slack at the interface joint.

Optionally, the cable management structure is securable to the feed cable that feeds into the second inlet. This may be particularly useful in embodiments where the second inlet rotates with the torquer portion (rather than being on an independently rotatable branched portion), since the cable management structure can help prevent the feed cable at the second inlet from tightening around the interface joint during rotation of the torquer portion.

The present disclosure also provides a system or a kit of parts including the interface joint described above in combination with a torque transfer unit attachable to the single cable assembly such that rotation of the torque transfer unit effects rotation of the single cable assembly. The torque transfer unit may be usable to transfer a user's rotating force to the flexible shaft of the single cable assembly connected to the electrosurgical instrument. By providing a torque transfer unit in combination with an interface joint as discussed herein, a surgeon may use the torque transfer unit to control the rotation of the instrument tip, while an assistant may hold onto the first (handle) portion of the interface joint, without in turn obstructing the rotation that is being effected by the surgeon via the torque transfer unit. Since the first portion of the housing can rotate independently from the second portion (which will rotate along with the distal torque transfer unit, if present, due to the attachment to the single cable assembly), the first portion of the interface joint can be conveniently held without inhibiting the rotation which the surgeon is attempting to cause. The torque transfer unit may be attachable to the single cable assembly at a position distally of the housing (i.e. nearer an entrance of the scoping device). As such, the torque transfer unit may be spaced from the housing in use.

As used herein, the torque transfer unit may alternatively be referred to as a ‘distal torquer’ or ‘distal torque transfer unit’, whereas the torquer portion of the interface joint may alternatively be referred to as a ‘proximal torquer’, ‘proximal torque transfer unit’, or ‘integrated torque transfer unit’.

In variant embodiments, the interface joint may be provided without a separate distal torque transfer unit. In such arrangements, the torquer portion of the interface joint may be used to control rotation of the instrument tip.

The torque transfer unit may be a torque transfer unit as described in GB 2 523 246 A, which is incorporated herein by reference in its entirety. Accordingly, the torque transfer unit may comprise an elongate clamp arranged to impart a gripping force along a length of the flexible sleeve, the elongate clamp comprising: an upper elongate housing member, a lower elongate housing member pivotably connected to the upper elongate housing member and defining a passage for the flexible sleeve, wherein the upper elongate housing member and the lower elongate housing member are pivotable between a release position in which the torque transfer unit is slidable up and down the flexible sleeve, and a clamping position, in which the flexible sleeve is gripped between the upper elongate housing member and the lower elongate housing member.

The invention includes the combination of the aspects and preferred features described except where such a combination is clearly impermissible or expressly avoided.

### SUMMARY OF THE FIGURES

Example embodiments illustrating the principles of the invention will now be discussed with reference to the accompanying figures in which like numerals denote like elements.

FIG. 1 is a schematic view of a complete electrosurgery system in which the present invention may be applied.

FIG. 2 is a side view of a system having an interface joint and torque transfer unit according to an embodiment of the invention;

FIG. 3 is a perspective exploded view of the interface joint shown in FIG. 2;

FIG. 4 is a side cutaway view of the interface joint shown in FIG. 2;

FIG. 5 is a side view of the interface joint shown in FIG. 2;

FIG. 6 is a side view of an interface joint according to a further embodiment of the invention;

FIG. 7 is a side view of an interface joint according to a further embodiment of the invention;

FIG. 8 is a side cutaway view of the interface joint shown in FIG. 7, omitting the fluid feed cable and single cable assembly;

FIG. 9 is a perspective view of the branched portion of the interface joint shown in FIG. 7;

FIG. 10 is a perspective cutaway view of the branched portion shown in FIG. 9;

FIG. 11 is a perspective exploded view of the branched portion shown in FIG. 9; and

FIG. 12 is a side view of the interface joint of FIG. 2 with a cable management structure.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

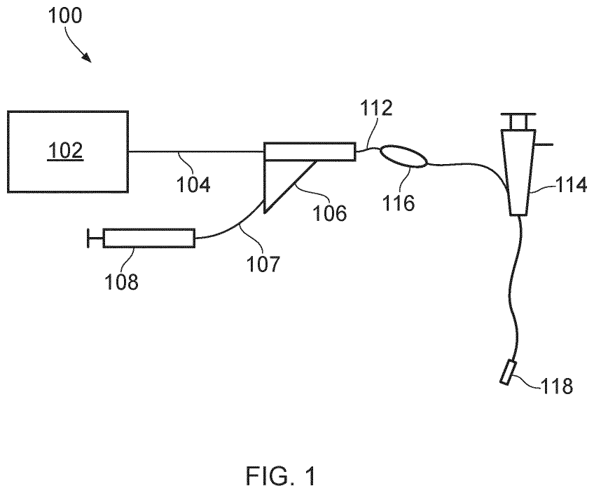

FIG. 1 is a schematic diagram of a complete electrosurgery system 100 that is capable of selectively supplying to the distal end of an invasive electrosurgical instrument any or all of RF energy, microwave energy and fluid, e.g. saline or hyaluronic acid. The system 100 comprises a generator 102 for controllable supplying electromagnetic (EM) energy. In the present embodiment, the EM energy includes RF EM energy and/or microwave frequency EM energy. A suitable generator for this purpose is described in WO 2012/076844, which is incorporated herein by reference.

The generator 102 is connected to an interface joint 106 by an interface cable 104. The interface joint 106 is also connected to receive a pressurised fluid supply from a fluid delivery apparatus 108 via a fluid supply cable 107. The function of the interface joint 106 is to combine the inputs from the generator 102 and fluid delivery device 108 into a single flexible shaft 112, which extends from the distal end of the interface joint 106. In this embodiment, the electrical inlet (for receiving EM energy from the generator 102) is in-line with an outlet to the flexible shaft 112, and the fluid inlet (for receiving fluid from the fluid supply cable 107) is angled relative to the outlet to the flexible shaft 112. It is to be understood that the shaft 112 may form part of the interface joint 106. The configuration of the interface joint 106 is discussed in more detail below.

The flexible shaft 112 is insertable through the entire length of an instrument (working) channel of a surgical scoping device 114. A torque transfer unit 116 may be mounted on a proximal length of the shaft 112 between the interface joint 106 and surgical scoping device 114. If present, the torque transfer unit 116 engages the shaft to permit it to be rotated within the instrument channel of the surgical scoping device 114.

The flexible shaft 112 has an electrosurgical instrument tip 118 that is shaped to pass through the instrument channel of the surgical scoping device 114 (e.g. an endoscope) and protrude (e.g. inside the patient) at the distal end of the instrument channel. The instrument tip includes an active tip for delivering RF EM energy and/or microwave EM energy into biological tissue and an aperture for delivering pressurised fluid (e.g. saline, Gelofusine, and/or hyaluronic acid with added marker dye). These combined technologies provide a unique solution for cutting and destroying unwanted tissue and the ability to seal blood vessels around the targeted area. By applying pressure to the fluid, the surgeon is able to inject the fluid between tissues layers in order to distend and mark the position of a lesion to be treated. The injection of fluid in this manner lifts and separates the tissue layers making it both easier to resect around the lesion and plane through the submucosal layer, reducing the risk of bowel wall perforation and unnecessary thermal damage to the muscle layer.

The instrument tip 118 further includes a protective hull positioned under the active tip to assist a tissue planing type resection action, again helping to protect against inadvertent perforation and ensure viability of the remaining tissue, which in turn facilitates more rapid healing and post operation recovery.

The structure of the instrument tip 118 may be particularly designed for use with a conventional steerable flexible endoscope having a working channel with an internal diameters of at least 2.2 mm and a working length of between 60 cm and 170 cm. As such the majority of the comparatively small diameter instrument is housed within the lumen of a much larger and predominantly polymer insulating device, i.e. the flexible endoscope channel. In practice, only 5 mm to 25 mm of the distal assembly protrudes from the distal end of the endoscope channel, in order not to block the field of view or adversely affect camera focussing. The protruding part of the distal assembly is the only portion of the instrument that ever makes direct contact with the patient.

At the proximal end of the endoscope working channel, which is typically held 50 cm to 80 cm from the patient, the flexible shaft 112 emerges from the working channel port and extends a further 30 cm to 100 cm to the interface joint 106. In use, the interface joint 106 is typically held by a gloved assistant throughout the procedure. The interface cable 104 is connected to the generator 102 using a QMA-type coaxial interface, which is designed to allow continuous clockwise or counter clockwise rotation. This permits the interface joint 106 to rotate with the torque transfer unit 116 under the control of the user. The assistant supports the interface joint 106 throughout the procedure in order to assist the user with sympathetic instrument rotation and fluid injection.

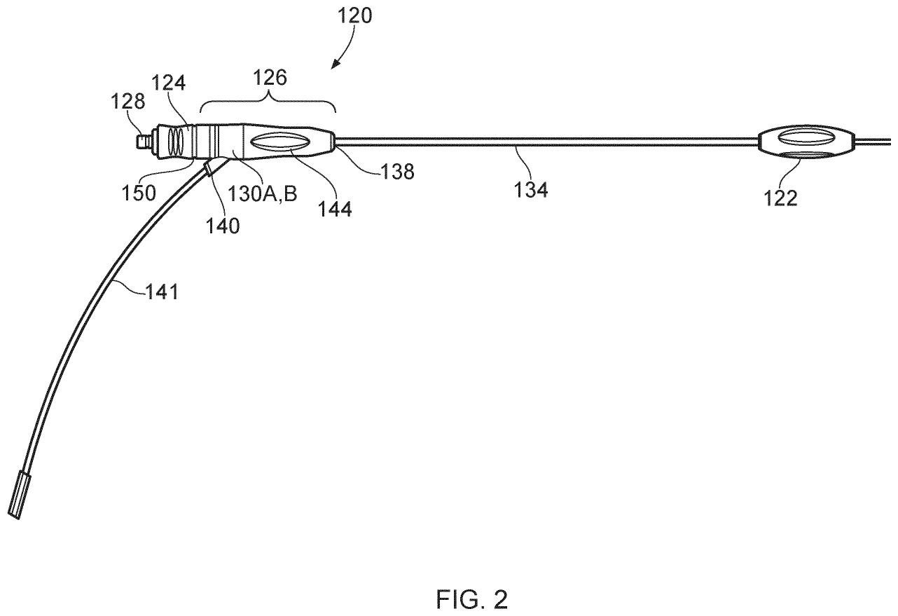

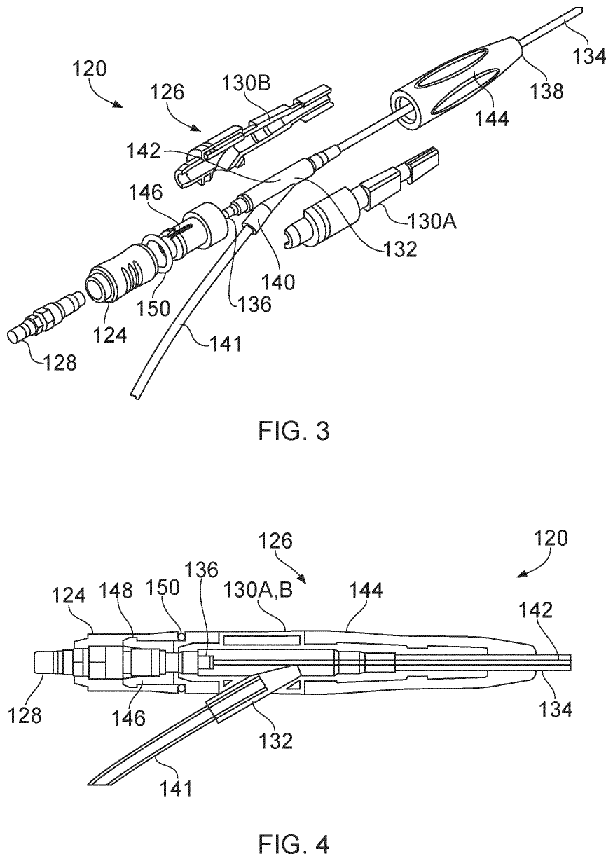

FIG. 2 is a side view showing a system comprising an embodiment interface joint 120 and a distal torque transfer unit 122. This system may be applied to the complete electrosurgery system 100 of FIG. 1. FIGS. 3, 4, and 5 show exploded, cross-sectional, and side views of the interface joint 120, respectively.

The interface joint 120 has a housing (or shell) which includes a first portion 124 at a proximal end and a second portion 126 at a distal end. The housing may comprise an electrically insulating material (e.g. plastic). The housing at least partially encases several internal components, as is best shown in FIGS. 3 and 4.

The first portion 124 comprises a first inlet in the form of an electrical inlet for receiving radiofrequency (RF) electromagnetic (EM) energy and/or microwave frequency EM energy from an electrosurgical generator (e.g. the electrosurgical generator 102 of FIG. 1). In this embodiment, the electrical inlet comprises a free-rotating connector 128 (e.g. MPX connector) which permits rotation of a coaxial feed cable (e.g. the interface cable 104 of FIG. 1) relative to the first portion 124.

As can be seen from FIG. 3, the second portion 126 comprises an outer casing formed of two releasably attachable (e.g. via snap-fit or push-fit) elongate casing elements 130A, 130B that are configured to mate to encapsulate a conduit 132. This arrangement provides a double isolation barrier as discussed above. The conduit 132 defines a branched passageway within which the fluid and EM inputs are integrated into a single cable assembly 134. The single cable assembly 134 is attached at its distal end to an instrument tip (e.g. the instrument tip 118 of FIG. 1).

The branched passageway of the conduit 132 has a first length and a second length. The first length provides an in-line connection between an electrical port 136 (for connecting to the electrical inlet 128) and an outlet 138 (for connecting to the single cable assembly 134). The second length extends at an angle to the first length and terminates in a second inlet. In this embodiment, the second inlet is a fluid inlet 140 for receiving fluid from a fluid supply (e.g. from the fluid delivery apparatus 108). The fluid inlet 140 is connected to a fluid feed cable 141.

In this embodiment, the second length extends at an acute angle to the first length, to form a Y-shaped conduit 132. Accordingly, the housing is in the shape of a pistol, i.e. it has an upper barrel portion and a lower adjoining portion which extends away from a proximal end of the upper barrel portion at an oblique angle.

The single cable assembly 134 comprises a flexible shaft which conveys a coaxial cable 142 and a fluid channel therethrough. The coaxial cable 142 is connected to the electrical inlet 128, and the fluid channel is in fluid communication with the fluid inlet 140 through the conduit 132. The fluid feed cable 141 may be used to convey pressurised fluid to the interface joint. The fluid channel may be configured to convey the pressurised fluid to the instrument tip, for piercing or lifting tissue without requiring the use of a needle. Accordingly, the interface joint 120 does not require a needle actuator and push rod for controlling a needle, and may therefore be relatively compact.

The second portion 126 is configured to attach to (e.g. clamp onto) the flexible shaft of the single cable assembly 134 and is operable such that rotation of the second portion effects rotation of the single cable assembly 134. The second portion 126 includes a gripping element 144 which is configured to overlie the casing elements 130A, 130B and which has a gripping surface thereon to facilitate grip by a user. In this embodiment, the gripping element 144 has a gripping surface that comprises a series of (e.g. four) longitudinally extending grooves.

Similarly, the first portion 124 may also include a gripping surface to facilitate grip by a user. For example, in this embodiment, the first portion 124 has a concave profile (when viewed from the side, e.g. as in FIG. 4) which is sized to be gripped between the thumb and forefinger of the operator. Additionally, the first portion 124 has a series of circumferentially-extending ribs and protrusions that extend at least partially around the circumference of the first portion.

The first portion 124 is connected to the second portion 126 by a free-rotating connector. Accordingly, the first portion 124 (which comprises the electrical inlet 128) is rotatable relative to the second portion 126 (which comprises the fluid inlet 140 and outlet 138). For example, in this embodiment, the first portion 124 is connected to the second portion 126 by rotatable snap-fit engagement features. In particular, as shown in FIG. 4, the second portion 126 includes a snap-fit engagement structure 146 (e.g. a male snap-fit engagement structure) which is attachable to the elongate casing elements 130A, 130B and is configured to mate to a corresponding snap-fit engagement structure 148 (e.g. a female snap-fit engagement structure) in the first portion 124. Additionally, the free-rotating connector may include a friction-reducing element 150 (e.g. an O-ring such as a PTFE O-ring) at the interface between the first portion 124 and second portion 126. This may help reduce any friction that could otherwise arise during relative rotation of the first and second portions.

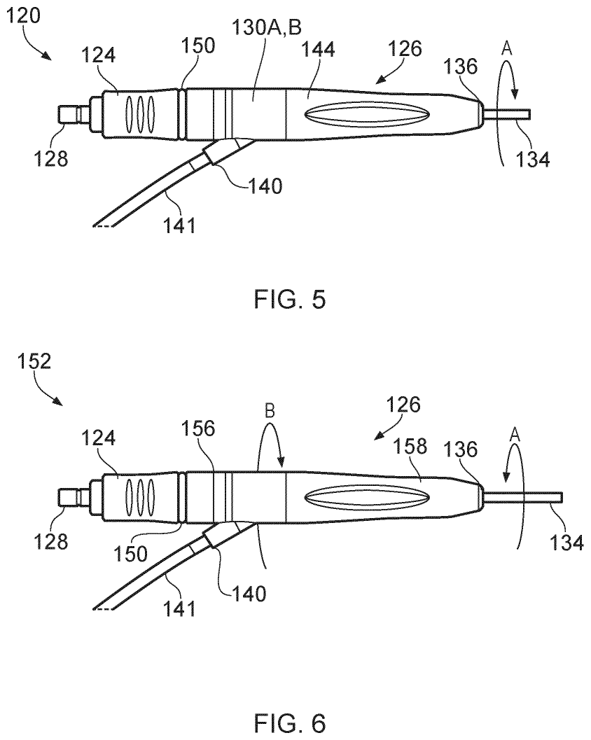

As shown in FIG. 5, in use, the second portion 126 may be rotated in a direction indicated by arrow A. Meanwhile, the first portion 124 may remain static. The second portion 126 can therefore be rotated to effect rotation of the single cable assembly 134, while the first portion 124 may be held without in turn obstructing rotation of the instrument tip. Accordingly, the first portion 124 may also be referred to as a handle portion, and the second portion 126 may be referred to as a torquer portion.

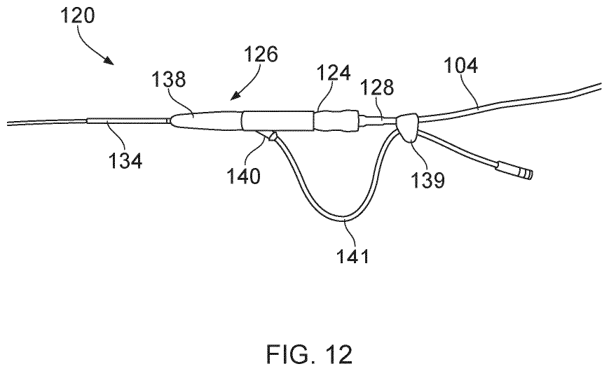

Since the fluid inlet 140 and electrical inlet 128 are at independently rotatable first and second portions 124 and 126, the fluid feed cable 141 may be less prone to tangling with the electrical feed cable. However, in some circumstances, during rotation of the second portion 126 (e.g. in the direction A), the fluid feed cable 141 may coil-up, wrapping around the housing. Therefore, optionally, the interface joint 120 may further include a cable management structure (e.g. a clip) 139 for securing a section of the fluid feed cable 141 to the interface joint, as shown in FIG. 12. This may help to secure excess slack in the cable, and to in turn mitigate excess friction being caused by the fluid feed cable 141 tightening around the housing during rotation of the instrument tip. For example, as shown in FIG. 12, the cable management structure 139 may secure the fluid feed cable 141 to a coaxial feed cable (e.g. interface cable 104).

FIG. 6 shows an interface joint 152 according to another embodiment of the invention. The interface joint 152 is similar to the interface joint 120, with similar reference numerals denoting similar elements, except where discussed otherwise.

The interface joint 152 of FIG. 6 differs from the interface joint 120 of FIGS. 2 to 5 in that the second portion 154 of the interface joint 152 includes an independently rotatable branched portion 156 and torquer portion 158. The fluid inlet 140 is part of the branched portion 156, and the outlet 138 is part of the torquer portion 158. Each of the first portion 124, branched portion 156, and torquer portion 158 are rotatable relative to each other. As shown in FIG. 6, in use, for example, the torquer portion 158 and shaft 134 may together be rotated in direction A, while the branched portion 156 may independently rotate in direction B, and the first (handle) portion 124 may remain static.

Advantageously, this arrangement allows each cable (the fluid feed cable 141, EM energy feed cable, and flexible shaft 134) to be independently orientated. For example, the fluid feed cable 141 may remain static during rotation of the single cable assembly 134, without affecting the EM feed cable. Accordingly, this arrangement may reduce any risk of the cables tangling and/or wrapping around the interface joint 152, and may obviate any need for a cable management structure.

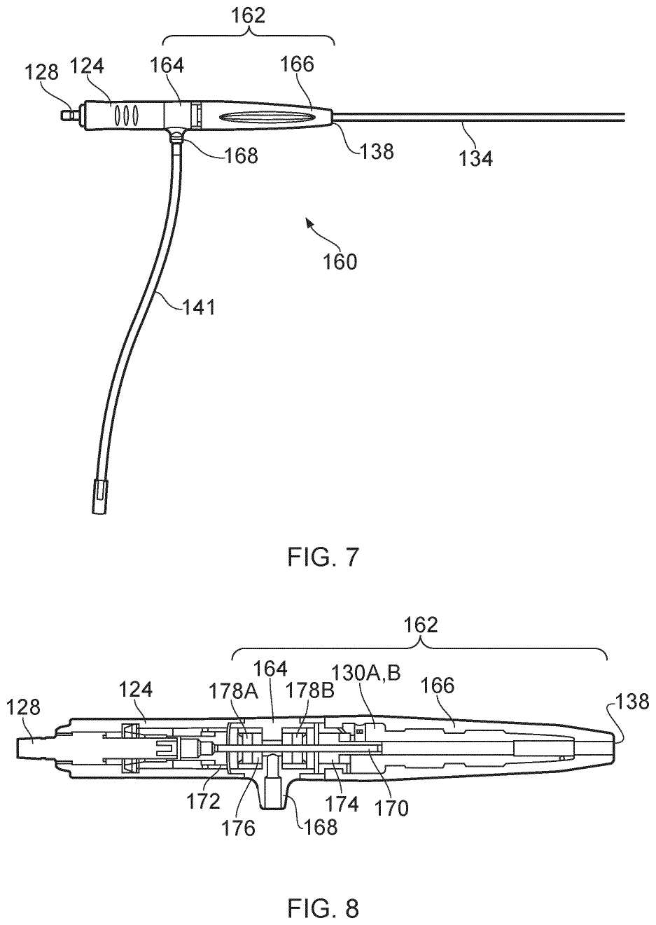

FIG. 7 shows an alternative interface joint 160 according to another embodiment of the invention. The interface joint 160 is similar to the interface joint 152 of FIG. 6, with similar reference numerals denoting similar elements, except where discussed otherwise.

FIG. 8 shows a cutaway view of the interface joint 160 of FIG. 7, omitting the fluid feed cable 141 and single cable assembly 134.

Similarly to the interface joint 152 of FIG. 6, the interface joint 160 of FIGS. 7 and 8 includes a second portion 162 having an independently rotatable branched portion 164 and torquer portion 166. In use, the branched portion 164 may remain stationary (thereby avoiding twisting of the fluid feed cable 141), while the torquer portion is axially rotated (relative to the branched portion 164) to rotate the cable assembly 134.

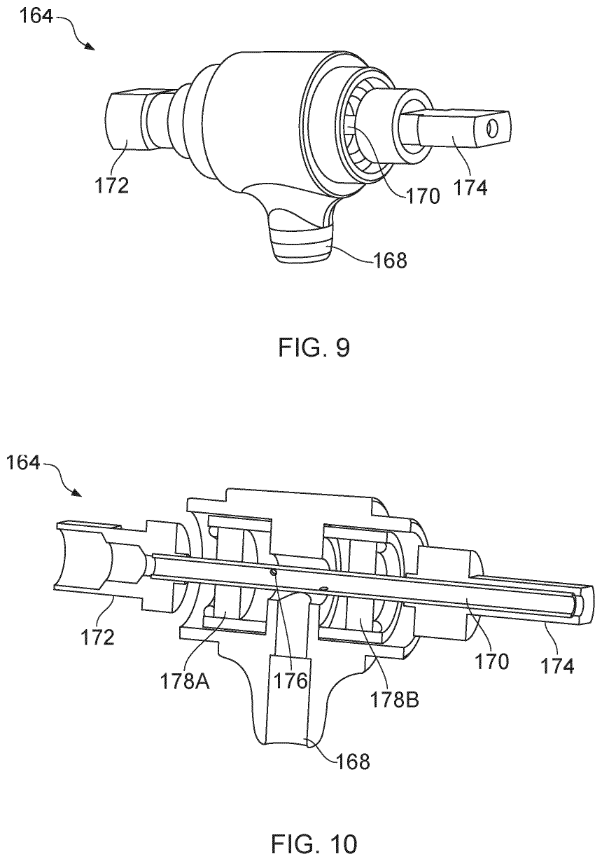

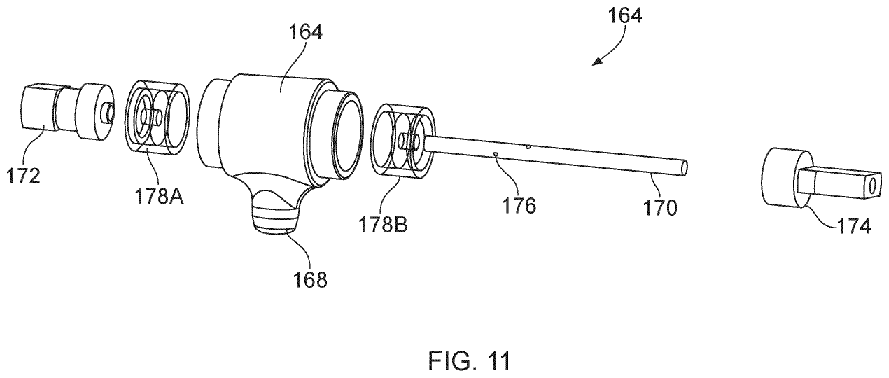

The interface joint 160 of FIGS. 7 and 8 differs from the interface joint 152 of FIG. 6 in the configuration of the branched portion. FIGS. 9 to 11 show perspective, cross-sectional, and exploded views of the branched portion 164, respectively.

The branched portion 164 comprises a T-shaped conduit defining a branched passageway having a first length (in-line with the outlet 138) and a second length perpendicular to the first length. Similarly to the previous embodiments, the second length terminates in a fluid inlet 168. A tubular member in the form of a hypotube 170 extends through the branched portion 164 along the first length.

At its proximal end, the branched portion 164 includes an electrical port 172 for connecting the hypotube 170 to the electrical inlet 128. At its distal end, the branched portion 164 includes an outlet port 174 for coupling the hypotube 170 to the single cable assembly 134. Accordingly, a coaxial cable (not shown) may connect to the electrical inlet 128, pass through the hypotube 170, and extend through the single cable assembly.

The hypotube has one or more apertures 176 (e.g. two apertures) for permitting fluid to flow into the hypotube from the fluid inlet 168. A first bung 178A and a second bung 178B are mounted within the T-shaped conduit to seal a fluid-tight cavity around the apertures 176 of the hypotube 170. The bungs may also be referred to as seal inserts or sealing bungs. The bungs 178A and 178B are rotatably fixed relative to the housing. The hypotube 170 is rotatable relative to and within each bung 178A and 178B. In this manner, the hypotube 170 may rotate relative to the fluid inlet 168 and electrical inlet 128.

In use, fluid from the fluid inlet 168 may flow into the fluid-tight cavity of the branched portion 164 and may enter the hypotube 170 through the one or more apertures 176. The fluid may flow within the hypotube (around the coaxial cable) and may then be conveyed into the single cable assembly 134.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

For example, in some embodiments, which are not illustrated, a connector arrangement comprising a hypotube and bungs as described above in connection with FIGS. 9 to 11 could alternatively be implemented in an arrangement where the torquer portion and branched portion are not independently rotatable (e.g. as in FIGS. 3 to 5).

Alternatively or additionally, in some embodiments, any of the components that form the housing of the interface joint (e.g. the first portion, branched portion, or torquer portion) can be modified to have multiple casing elements that are connected together, in a similar manner to the casing elements 130A,B e.g. via push-fit or snap-fit connections. For example, in some embodiments, the first portion 124 and/or branched portion 164 may each be modified to comprise multiple (e.g. two) releasably attachable/matable casing elements (e.g. two half-shells). By configuring one or more portions of the housing in this manner, the assembly of the interface joint can be made more convenient.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### REFERENCE NUMERALS

100 electrosurgery system
102 generator
104 interface cable
106 interface joint
electrosurgical instrument
112 flexible shaft
118 instrument tip
107 fluid supply cable
114 surgical scoping device
116 torque transfer unit
108 fluid delivery apparatus
120, 152, 160 interface joint
124 first portion
128 first inlet (e.g. electrical inlet)
148 snap-fit engagement structure
150 o-ring
126, 154, 162 second portion
130A,B casing elements
146 snap-fit engagement structure
156 branched portion
132 conduit
136 electrical port
140 second inlet (e.g. fluid inlet)
141 fluid feed cable
139 cable management structure
164 branched portion
168 fluid inlet
170 tubular member (e.g. hypotube)
176 apertures
172 electrical port
174 outlet port
178A,B bungs
158, 166 torquer portion
144 gripping element
138 outlet
134 single cable assembly
142 coaxial cable
122 torque transfer unit

1. An interface joint for interconnecting an electrosurgical generator, a fluid supply, and an electrosurgical instrument, the interface joint comprising:
a housing having:
an electrical inlet for receiving radiofrequency (RF) electromagnetic (EM) energy and/or microwave frequency EM energy from the electrosurgical generator;
a fluid inlet for receiving fluid from the fluid supply; and
an outlet; and
a single cable assembly for connecting the outlet to an instrument tip of the electrosurgical instrument, the single cable assembly comprising a flexible shaft having:
a coaxial cable that is connected to the electrical inlet; and
a fluid channel that is in fluid communication with the fluid inlet;
wherein the housing includes a first portion and a second portion, the second portion being attached to the single cable assembly such that rotation of the second portion effects rotation of the single cable assembly; and the first portion being rotatable relative to the second portion;
wherein the first portion includes a first inlet and the second portion includes a second inlet, wherein the first inlet is one of the fluid inlet or electrical inlet, and the second inlet is the other of the fluid inlet or electrical inlet.

2. The interface joint of claim 1, further comprising a fluid feed cable connected to the fluid inlet for conveying fluid from the fluid supply to the interface joint.

3. The interface joint of claim 1, wherein the second portion comprises a conduit defining a branched passageway, the branched passageway having a first length in line with the first inlet and the outlet, and a second length having the second inlet.

4. The interface joint of claim 3, wherein the second length extends at an angle to the first length.

5. The interface joint of claim 4, wherein the second length extends at a substantially perpendicular angle to the first length.

6. The interface joint of claim 1, wherein the first inlet is the electrical inlet, and the second inlet is the fluid inlet.

7. The interface joint of claim 1, wherein the second portion includes a branched portion and a torquer portion, the branched portion being rotatable relative to the torquer portion, wherein the branched portion includes the second inlet and the torquer portion includes the outlet.

8. The interface joint of claim 7, wherein the branched portion is configured to combine the fluid and EM energy into a tubular member to be conveyed to the torquer portion; and
wherein the tubular member is rotatable relative to the branched portion.

9. The interface joint of claim 8, further including one or more bungs within the branched portion to form a fluid-tight seal around the tubular member.

10. The interface joint of claim 7- or 8, wherein the tubular member is configured to convey the coaxial cable that is connected to the electrical inlet, and the tubular member includes one or more apertures for permitting fluid to flow into the tubular member.

11. The interface joint of claim 1, wherein the interface joint does not include a needle actuator on the housing, the needle actuator being attached to a push rod that extends out of the housing through the outlet for controlling a needle at the distal end of the instrument.

12. The interface joint of claim 1, wherein two or more of the portions of the housing are releasably attachable to each other.

13. The interface joint of claim 1, further comprising:
a cable management structure for securing a section of a feed cable to the interface joint, the feed cable being either:
a fluid feed cable for conveying fluid to the fluid inlet from the fluid supply; or
an EM feed cable for conveying the EM energy from the electrosurgical generator to the electrical inlet.

14. The interface joint of claim 13, wherein the feed cable is connected to the second inlet.

15. A kit of parts including:
the interface joint of claim 1; and
a torque transfer unit attachable to the single cable assembly such that rotation of the torque transfer unit effects rotation of the single cable assembly.
